(19) United States
(12) Patent Application Publication
(10) Pub. No.: US 2010/0196370 A1
(43) Pub. Date: Aug. 5, 2010

(54) FUSION PROTEIN OF IMMUNOGLOBULIN FC AND HUMAN APOLIPOPROTEIN(A) KRINGLE FRAGMENT

(75) Inventors: Hyun-Kyung Yu, Gyeonggi-do (KR); Yeup Yoon, Gyeonggi-do (KR); Jin-Hyung Ahn, Gyeonggi-do (KR); In-Hwan Lim, Gyeonggi-do (KR); Ho-Jeong Lee, Gyeonggi-do (KR); Jang-Seong Kim, Gyeonggi-do (KR); Doo-Hong Park, Seoul (KR)

Correspondence Address: MOORE & VAN ALLEN PLLC, P.O. BOX 13706, Research Triangle Park, NC, 27709, US

(73) Assignee: MOGAM BIOTECHNOLOGY RESEARCH INSTITUTE, Gyunggi-do (KR)

(21) Appl. No.: 12/519,653

(22) PCT Filed: Nov. 16, 2007
(86) PCT No.: PCT/KR2007/005790

§ 371 Date: Apr. 5, 2010

(30) Foreign Application Priority Data
Dec. 21, 2006 (KR) 10-2006-0131777

(51) Int. Cl. A61K 39/395; C07K 16/00; C07H 21/04; C12N 15/63; C12N 5/10; C12P 21/06; A61P 35/04

(52) U.S. Cl. 424/134.1; 530/387.3; 536/23.4; 435/320.1; 435/375; 435/358; 435/69.6

(57) ABSTRACT

The present invention relates to an LK8-Fc fusion protein, which has increased angiogenesis inhibitory activity and in vivo stability. More specifically, relates to an LK8-Fc fusion protein in which an LK8 protein having angiogenesis inhibitory activity is fused with the Fc region of human immunoglobulin IgG1, as well as a composition for treating cancer, which contains the fusion protein. The LK8-Fc fusion protein has not only angiogenesis inhibitory activity leading to anticancer and metastasis inhibitory activities, but also a very long in vivo half-life, and thus can be used as a more efficient and economic cancer therapeutic agent or cancer inhibitor.

### TECHNICAL FIELD

The present invention relates to an LK8-Fc fusion protein, which has increased angiogenesis inhibitory activity and in vivo stability, and more specifically, to an LK8-Fc fusion protein in which an LK8 protein having angiogenesis inhibitory activity is fused with the Fc region of human immunoglobulin IgG1, as well as a composition for treating cancer, which contains the fusion protein.

### BACKGROUND ART

Angiogenesis refers to the process by which new blood vessels are formed from pre-existing vessels. It is known that, in normal physiological conditions, vascular endothelial cells are maintained in a state in which they are hardly proliferated and angiogenesis occurs only in extremely limited cases including a woman's menstrual cycle. A failure in controlling the mechanism of angiogenesis can cause many pathological diseases including cancer, diabetic retinopathy, rheumatoid arthritis, psoriasis, etc. In the case of tumors, it is known that, although cancer can grow to a volume of a few mm³ without the help of blood vessels, angiogenesis is essential in order for cancer to N. Eng. J. Med., 333:1757, 1995; Folkman, J., New Engl. J. Med., 285:1182, 1971).

For the initiation of tumor angiogenesis, two conditions, that is, an increase in angiogenesis promoting factors and a decrease in angiogenesis inhibitory factors, should be satisfied. A typical example of endogenous angiogenesis inhibitors is angiostatin. It was reported that angiostatin is a portion of plasminogen, an enzyme associated with blood clotting, consists of kringle structures, and has the ability to inhibit angiogenesis in in vitro and in vivo conditions (O'Reilly, M. S. et al., Cell, 79:315, 1994). The kringles are structural domains of proteins consisting of about 80 amino acids and three intramolecular disulfide bonds and constitute an independent folding unit. The kringle structures are found in many proteins such as prothrombin, urokinase, hepatocyte growth factor, and apolipoprotein(a). Peculiarly, it has been reported that various kringles, such as prothrombin kringle and urokinase kringle, show the ability to inhibit angiogenesis (Lee, T. H. et al., J. Biol. Chem., 273:28805, 1998; Kim et al., J. Biol. Chem., 278:11449, 2003).

Glycoprotein apolipoprotein(a) covalently bonds with apo B-100, which is the major protein component of low-density lipoprotein (LDL), to form lipoprotein (a) (Fless, G. M., J. Biol. Chem., 261:8712, 1986). It is known that lipoprotein (a) is involved in cholesterol transport in vivo, and an increase in the concentration of lipoprotein (a) in plasma is associated with artherosclerosis and heart diseases (Armstrong, V. W. et al., Artherosclerosis, 62:249, 1986; Assmann, G., Am. J. Cardiol., 77:1179, 1996). Apolipoprotein(a) includes two types of kringle domains, which show homology to plasminogen kringles IV and V, and an inactive protease-like domain. The apolipoprotein(a) kringle IV-like domain is divided into 10 subtypes (IV1-IV10) according to amino acid sequence homology, and each of them has only one copy except for IV2 kringle which has 3-42 copy numbers in various human alleles of the apolipoprotein(a) gene. The last kringle V has an amino acid sequence homology of 83.5% with plasminogen kringle V.

The present inventors observed that a portion of kringles constituting apolipoprotein(a) (kringle KV38; hereinafter, referred to as “LK8” protein”) had the ability to inhibit angiogenesis in in vitro and in vivo conditions, which resulted in anticancer and metastasis inhibitory actions (WO 2001/019868, entitled “Angiogenesis inhibitor comprising LK6, LK7, LK8 and LK68”; WO 2004/073730, entitled “Anticancer agent containing LK8”). However, the LK8 protein was found to have an in vivo half-life of only 7-11 hours in monkeys, and thus it has a problem in that it needs to be repeatedly administered at short intervals in order to exhibit anticancer efficacy. Also, angiogenesis inhibitors are usually cytostatic rather than cytotoxic and must be continuously administered for a long period of time in order to exhibit anticancer effects (Jain, R. K. et al., Nat. Clin. Pract. Oncol., 3:24, 2006).

Accordingly, for a long-term continuous administration, the production of a large amount of LK8 recombinant proteins and the resulting increase in production cost are required, and high treatment cost and long-term treatment period impose a heavy burden on patients. For this reason, it is technically difficult to develop anticancer agents using the LK8 protein.

Meanwhile, the preparation of a fusion protein of immunoglobulin or its fragment with an active protein has been performed for an increase in antigenicity, the easiness of purification, an increase in blood half-life, etc. Examples thereof include: an interleukin receptor, which is a fusion protein of a protein drug, which has both the function of an immunoglobulin fragment itself and the function of useful protein, with immunoglobulin Fc (Korean Patent 249572); a fusion protein obtained by fusing INF-α with Fc so as to increase the blood half-life of INF-α, etc. However, the fusion protein of INF-α and Fc has very increased half-life, but is disadvantageous in that the activity of INF-α is reduced (U.S. Pat. No. 5,723,125).

Accordingly, the present inventors have made many efforts to find a method which enables the LK8 protein to maintain a long half-life while the angiogenesis inhibitory effect thereof is not reduced, when the LK8 protein is administered in vivo. For this purpose, the present inventors have constructed an LK8-Fc fusion protein by fusing the Fc region of IgG1 to the C-terminal end of the LK8 protein and examined the effect thereof. As a result, the present inventors have found that the fusion protein shows a completely unexpected effect in that the half-life of the fusion protein is increased by about 40-50 fold compared to that of the LK8 protein due to the Fc fusion partner without influencing the effect of the LK8 protein itself, thereby completing the present invention.

### SUMMARY OF THE INVENTION

It is an object of the present invention to provide an LK8-Fc fusion protein, which has increased bioavailability by fusing the Fc region of human immunoglobulin IgG1 to the C-terminal end of the LK8 protein, which is the kringle fragment of human apolipoprotein(a) having anticancer and metastasis inhibitory effects, using gene fusion technology.

Another object of the present invention is to provide a composition for treating cancer, which contains the LK8-Fc fusion protein.

Still another object of the present invention is to provide a composition for inhibiting angiogenesis, which contains said LK8-Fc fusion protein.

To achieve the above objects, in one aspect, the present invention provides an LK8-Fc fusion protein in which a LK8 protein is fused with the Fc region of human immunoglobulin IgG1. In the present invention, the LK8-Fc fusion protein preferably contains an additional Igκ leader sequence for extracellularly secreting the fusion protein.

In another aspect, the present invention provides a gene encoding said LK8-Fc fusion protein, a recombinant vector containing said gene, and recombinant cells transfected with said recombinant vector. In the present invention, the cells are preferably animal cells. The animal cells are preferably CHO/LK8-Fc cells.

In still another aspect, the present invention provides a composition for treating cancer and a composition for inhibiting angiogenesis, which contain said LK8-Fc fusion protein. In the present invention, the cancer is preferably selected from the group consisting of colorectal cancer, pancreatic cancer, prostate cancer, renal cancer, melanoma, bone metastases of prostate cancer, and ovarian cancer.

Other features and aspects of the present invention will be apparent from the following detailed description and the appended claims.

### BRIEF DESCRIPTION OF DRAWINGS

FIG. 1 shows a process for constructing expression vector pMSG/LK8-Fc expressing a gene encoding an LK8-Fc fusion protein.

FIG. 2 is a graphic diagram showing the growth curve and cell viability of a CHO/LK8-Fc cell line in spinner culture.

FIG. 3 is a graphic diagram showing the elution of the LK8-Fc fusion protein as a function of glycine buffer concentration and time in a process of purifying the LK8-Fc fusion protein using affinity chromatography.

FIG. 4 shows the results of western blot analysis of the purified LK8-Fc fusion protein.

FIG. 5 is a graphic diagram showing the number of migrated cells per field according to sample treatment in a wound migration assay in which endothelial cells were treated with the LK8-Fc fusion protein.

FIG. 6 is a graphic diagram showing cell migration rate (%) according to sample treatment in a wound migration assay in which endothelial cells were treated with the LK8-Fc fusion protein.

FIG. 7 is a graphic diagram showing the in vivo angiogenesis inhibition of the LK8-Fc fusion protein in a CAM assay.

FIG. 8 is a graphic diagram showing the pharmacokinetic (PK) profile of the LK8-Fc fusion protein.

FIG. 9 is a graphic diagram showing the tumor growth inhibitory effect by the treatment with the LK8-Fc fusion protein.

FIG. 10 is a graphic diagram showing the metastasis inhibitory effect by the treatment with the LK8-Fc fusion protein.

### DETAILED DESCRIPTION OF THE INVENTION AND PREFERRED EMBODIMENTS

In the present invention, recombinant plasmid pMSG/LK8-Fc comprising a gene sequence encoding the LK8-Fc fusion protein was constructed, and a CHO/LK8-Fc cell line, which is transfected with the gene to produce a recombinant LK8-Fc fusion protein, was established. Also, mice were treated with the LK8-Fc fusion protein produced from the established CHO/LK8-Fc cell line and, as a result, it was observed that the fusion protein inhibited the growth and metastasis of the cancer.

Also, it was found that the half-life of the LK8-Fc fusion protein according to the present invention was increased by about 40-50 fold compared to the LK8 protein due to the Fc fusion partner without adversely affecting the LK8 protein itself, and thus showed the effect of reducing the total amount of protein administered and the frequency of administration.

This fact is considered to be an effect which cannot be predicted at all by those skilled in the art, because a conventional fusion protein of a useful protein with Fc shows an effect lower than that of the parent protein or shows an insufficient effect on an increase in half-life, whereas the LK8-Fc fusion protein of the present invention has the same effect as that of the prior LK8 protein and, at the same time, the half-life thereof is much longer than that of the prior LK8 protein.

In the present invention, it was observed that the LK8-Fc fusion protein inhibited the migration of human endothelial cells, induced by bFGF in in vitro conditions, and had a function of inhibiting angiogenesis in in vivo conditions. Meanwhile, it was observed that, when the LK8-Fc protein was administered once into the muscle of SD rats (6w, male, Charles River, Japan), the in vivo half-life thereof was increased by about 40-50 fold compared to that of the LK8 protein due to the Fc fusion partner. This suggests that the fusion protein can exhibit high efficacy, even when the administration frequency and dosage of the drug are reduced. Accordingly, the LK8-Fc fusion protein can show anticancer and metastasis inhibitory effects, even when it is administered in a dosage of less than 1/10 compared to that of the LK8 protein, once at intervals of a minimum of 7 days, as an effective amount. However, the administration frequency and dosage are not necessarily limited thereto and can be determined depending on the patient's age, sex and health condition, and the kind and severity of disease.

When the LK8-Fc fusion protein according to the present invention is used in combination with chemotherapy or radiotherapy, which have been used in the prior art, a synergistic effect can be obtained. Also, the fusion protein of the present invention can be used in combination with other kinds of formulations having an angiogenesis inhibitory effect. In addition, the LK8-Fc fusion protein of the present invention can be administered in combination with another angiogenesis inhibitor having a mechanism different from that of the inventive fusion protein, and in this case, effective anticancer or metastasis inhibition can be achieved.

Immunoglobulin heavy chain constant region comprises 4 or 5 domains, which consist of CH1-hinge-CH2-CH3(-CH4). The DNA sequences of the heavy chain domains have cross-homology among the immunoglobulin classes. For example, the CH2 domain of IgG is homologous to the CH2 domain of IgA and IgD, and to the CH3 domain of IgM and IgE.

As used herein, the term “Fc region” refers to the carboxyl terminal portion of an immunoglobulin chain constant region, preferably an immunoglobulin heavy chain constant region, or part thereof. For example, the immunoglobulin Fc region may comprise: (1) the CH1 domain, the CH2 domain and the CH3 domain; (2) the CH1 domain and the CH2 domain; (3) the CH1 domain and the CH3 domain; (4) the CH2 domain and the CH3 domain; or (5) a combination of two or more domains and the immunoglobulin hinge region. In a preferred embodiment of the present invention, the immunoglobulin Fc region at least comprises the immunoglobulin hinge region, the CH2 domain and the CH3 domain and lacks the CH1 domain.

A preferred class of immunoglobulin, from which the heavy chain constant region is derived, is IgG (Igγ) (γ subclass 1, 2, 3 or 4). Other classes of immunoglobulin IgA(Igα), IgD(Igδ), IgE(Igε) and IgM(Igμ) may be used in the present invention. The selection of a suitable immunoglobulin heavy chain constant region is described in detail in U.S. Pat. No. 5,541,087 and U.S. Pat. No. 5,726,044. It is considered that those skilled in the art can select a specific immunoglobulin heavy chain constant region sequence from specific immunoglobulin classes and subclasses in order to obtain specific results. The portion of the DNA encoding the immunoglobulin Fc region preferably comprises at least a portion of a hinge domain, and a portion of CH3 domain of Fcγ or the homologous domains in any of IgA, IgD, IgE, or IgM.

Depending on the intended use, a constant region gene derived from species (e.g., mice or rats) other than human may be used. The immunoglobulin Fc region which is used as a fusion partner in the DNA construct can be generally obtained from any mammalian species. When it is not undesirable to induce an immune response to the Fc region in host cells or animals, the Fc region may be derived from the same species as the host cells or animals. For example, if the host cells or animals are human beings, a human immunoglobulin Fc region may be used, and if the host cells or animals are mice, a rodent immunoglobulin Fc region may be used.

A nucleic acid sequence encoding a human immunoglobulin Fc region useful in the practice of the present invention and an amino acid sequence translated therefrom are set forth in SEQ ID NO. 2, but the scope of the present invention is not limited thereto. For example, it is possible to use other immunoglobulin Fc region sequences, such as those encoded by nucleotide sequences present in the GenBank or EMBL database, for example, AF045536.1 (Macaca fuscicularis), AF045537.1 (Macaca mulatta), AB016710 (Felix catus), K00752 (Oryctolagus cuniculus), U03780 (Sus scrofa), Z48947 (Camelus dromedarius), X62916 (Bos taurus), L07789 (Mustela vision), X69797 (Ovis aries), U17166 (Cricetulus migratorius), X07189 (Rattus rattus), AF57619.1 (Trichosurus vulpecula) or AF035195 (Monodelphis domestica).

Also, the substitution or deletion of amino acids in the immunoglobulin heavy chain constant region can be used in the practice of the present invention. For example, amino acid substitution can be introduced into the upper CH2 region in order to produce Fc mutants having a reduced affinity for Fc receptor (Cole et al., J. Immunol., 159:3613, 1997). Any person skilled in the art can prepare such constructs using well-known molecular biological techniques.

In the present invention, conventional recombinant DNA technology is used to produce an Fc fusion protein useful in the practice of the present invention. The Fc fusion construct is preferably produced at the DNA level, and the DNA thus produced is inserted into an expression vector and expressed, thus producing the fusion protein of the present invention.

As used herein, the term “vector” means any nucleic acid comprising a nucleotide sequence that is competent to be incorporated into a host cell and to be recombined with and integrated into a host cell's genome, or to replicate autonomously as an episome. Such vectors include linear nucleic acids, plasmids, phagemids, cosmids, RNA vectors, viral vectors and the like. Examples of viral vectors include retrovirus, adenovirus and adeno-associated virus, but the scope of the present invention is not limited thereto.

As used herein, the term “gene expression” or “expression of a target gene” refers to the transcription of DNA sequence, the translation of mRNA transcript, and the secretion of Fc fusion protein product.

An appropriate host cell can be transformed or transfected with the DNA sequence of the present invention, and utilized for the expression and/or secretion of a target protein. Preferred host cells for use in the present invention include immortal hybridoma cells, NS/O myeloma cells, 293 cells, Chinese hamster ovary (CHO) cells, Hela cells, and COS cells.

An expression system which has been used to produce a fusion protein at a high expression level in mammalian cells is a DNA construct encoding a secretion cassette, comprising, in the 5′ to 3′ direction, a signal sequence, a target protein and an immunoglobulin Fc region.

As used herein, the term “leader sequence” refers to a sequence which directs the secretion of the LK8-Fc fusion protein, and then is translated in host cells and cleaved. The leader sequence in the present invention is a polynucleotide encoding an amino acid sequence which initiates the transport of a protein across the membrane of endoplasmic reticulum. Leader sequences useful in the present invention include antibody light chain leader sequences, for example, antibody 14.18 (Gillies et al., J. Immunol. Meth., 125:191, 1989), Igκ leader sequences, antibody heavy chain signal sequences, for example, MOPC141 antibody heavy chain leader sequences (Sakano et al., Nature, 286:5774, 1980), and other leader sequences known in the art (Watson et al., Nucleic Acids Research, 12:5145, 1984).

The present invention provides a method of treating various cancers, viral diseases, related diseases and the causes thereof by administering the inventive LK8-Fc fusion protein to mammals having such diseases. The related diseases may include various solid cancers which proliferate and metastasize by angiogenesis, but the scope of the present invention is not limited thereto.

Cancer in the present invention may be colorectal cancer, pancreatic cancer, prostate cancer, renal cancer, melanoma, bone metastases of prostate cancer, and ovarian cancer, but the scope of the present invention is not limited thereto.

The composition of the present invention can be administered by any route suitable for a specific molecule. The inventive composition may be provided by any suitable means, directly (e.g., topically, as by injection, subcutaneous injection or topical administration to a tissue locus) or systematically (e.g., parenterally or orally). Where the composition is to be provided parenterally, such as by intravenous, subcutaneous, ophthalmic, intraperitoneal, intramuscular, buccal, rectal, vaginal, intraorbital, intracerebral, intracranial, intraspinal, intraventricular, intrathecal, intracisternal, intracapsular, intranasal or by aerosol administration, the composition preferably comprises part of an aqueous or physiologically compatible fluid suspension or solution. Thus, the carrier or excipient is physiologically acceptable so that in addition to delivery of the desired composition to the patient, it does not adversely affect the patient's electrolyte and/or volume balance. The fluid medium for the agent thus can comprise normal physiologic saline.

The dosage of the LK8-Fc fusion protein according to the present invention is preferably 0.03-300 mg/m², and more preferably 0.3-30 mg/m². However, the optimum dosage varies depending on the disease to be treated and the presence of side effects, but can be determined through conventional experiments. The administration of the fusion protein can be performed either by periodic bolus injections, or by intravenous or intraperitoneal administration from a reservoir which is external (e.g., an i.v. bag) or internal (e.g., a bioerodible implant). Also, the fusion protein of the present invention may be administered together with a plurality of different biologically active molecules to a target receptor. However, the optimal combination, mode of administration and dosage of the fusion protein and other molecules can be determined through conventional experiments by persons skilled in the art.

The angiogenesis inhibitor according to the present invention can be applied as agents for treating various lesions associated with angiogenesis, including various tumors and tumor metastasis, diabetic retinopathy, rheumatoid arthritis, psoriasis and the like. In this case, the LK8-Fc fusion protein according to the present invention may also be used in combination with other therapeutic agents associated with the relevant disease.

### EXAMPLES

Hereinafter, the present invention will be described in further detail with reference to examples. It will be apparent to one skilled in the art that these examples are for illustrative purpose only and are not construed to limit the scope of the present invention.

### Example 1

### Construction of Recombinant Vector Expressing LK8-Fc Fusion Protein

In order to construct a vector encoding a fusion protein of LK8 and Fc, LK8 gene (SEQ ID NO: 1) was obtained by PCR using, as a template, pET11B vector (WO 2001/019868) containing the LK8 gene, which is previously prepared by the present inventors. In addition, a gene (SEQ ID NO: 2) encoding Fc was obtained by PCR using, as a template, pRC13-Hpa vector (Korean Patent 467706). Primers used in each of the PCR reactions are shown in Table 1 below.

Specifically, the PCR reaction was performed in the following conditions: denaturation of the template DNA at 94° C. for 5 min, and then 30 cycles of 30 sec at 94° C., 30 sec at 56° C. and 1 min at 72° C., followed by extension at 72° C. for 5 min. Also, for easy cloning, restriction enzyme digestion sites were inserted into each of the primers, such that the resulting PCR products had the restriction enzyme digestion sites.

The two gene fragments, produced through the PCR reactions, were inserted into a pSecTag vector (Invitrogen, USA) containing an Igκ leader sequence for facilitating the extracellular secretion of a protein to be produced. Specifically, the LK8 gene fragment and the pSecTag vector were digested with SfiI and BamHI, and then the LK8 gene fragment was ligated to the pSecTag vector to construct pSecTag-LK8. The Fc gene fragment was digested with BamHI and XhoI, and then ligated to the pSecTag-LK8 digested with BamHI and XhoI, thus constructing pSecTag/LK8-Fc.

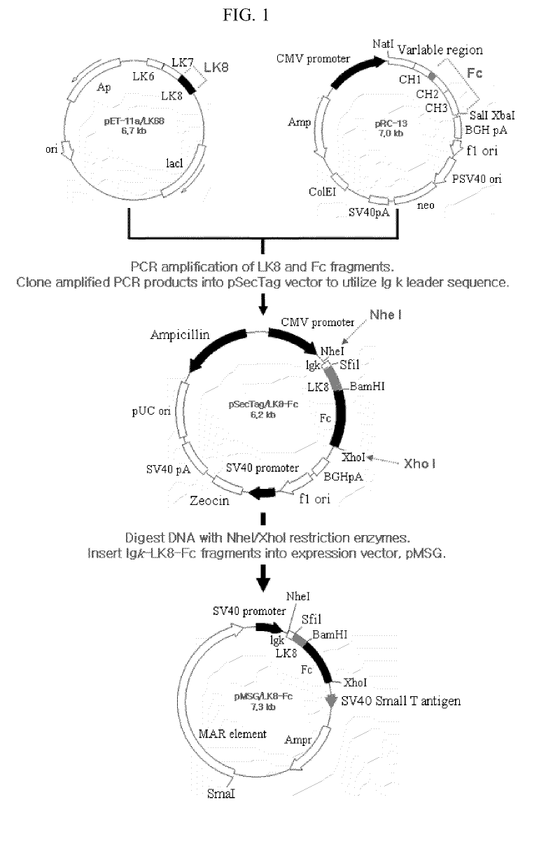

In the plasmid pSecTag/LK8-Fc, the Igκ leader sequence, the LK8 gene and the Fc gene were digested with restriction enzymes and inserted into mammalian cell expression vector pMSG (KCCM 10202; Korean Patent Publication 10-2002-0010327). That is, the pMSG vector and the pSecTag-LK8-Fc plasmid were digested with restriction enzymes NheI and XhoI, and then the digested Igκ-LK8-Fc fragment was inserted into the pMSG vector, thus constructing pMSG/LK8-Fc (FIG. 1).

TABLE 1
Primers used in construction of pMSG/LK8-Fc

| | |
|---|---|
| SEQ | ID NO: |

| | | |
|---|---|---|
| LK8 | 5′-GCGGCCCAGCCGGCCGAACAAGACTGTATGTTTG-3′ | 3 |
| LK8 antisense | 5′-CGGGATCCAGAGGATGCACAGAGAGGGATATC-3′ | 4 |
| Fc sense | 5′-CGGGATCCGAGCCCAAATCTTGTGAC-3′ | 5 |
| Fc antisense | 5′-TATACTCGAGTCATTTACCCGGAGACAGGG-3′ | 6 |

Underlines indicate restriction enzyme recognition sites

### Example 2

### Establishment of Animal Cell Line Expressing Large Amount of LK8-Fc Fusion Protein

In order to establish an animal cell line producing the LK8-Fc fusion protein, the pMSG/LK8-Fc, constructed in Example 1, together with the DHFR (dihydrofolate reductase) gene (Columbia University, USA), was transfected into DHFR gene-deleted cell line CHO DG44 (Columbia university, USA) using Dosper (Roche, Switzerland). Then, from the cell line, colonies adapted to a 10% serum-containing MEM-α minimal medium (GIBCO, USA) were primarily selected, and the selected colonies were subcultured by progressively increasing the concentration of MTX (Methotrexate; ChoongWae Pharma Corporation, Korea) (including 50 nM and 1 μM). During the subculture, among colonies showing tolerance to MTX, a cell line secreting a large amount of the target protein was secondarily selected. The selected cell line was cultured in a serum-free medium HyQ-SFM-CHO (Promega, USA)-containing spinner flask in order to facilitate the mass production of the protein, and the finally selected cell line was named “CHO/LK8-Fc”

### Example 3

### Purification of LK8-Fc Fusion Protein

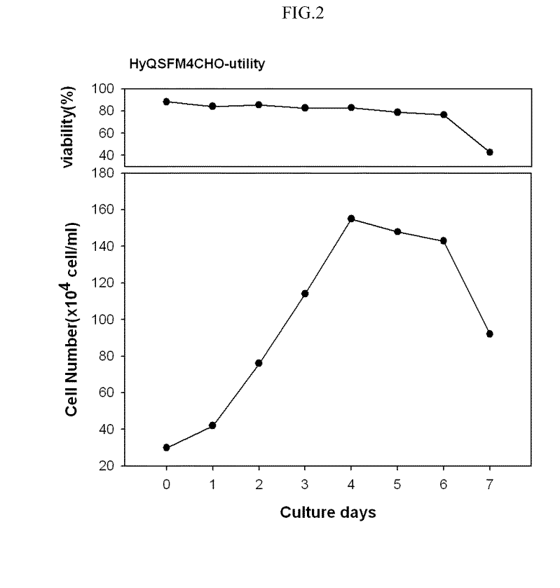

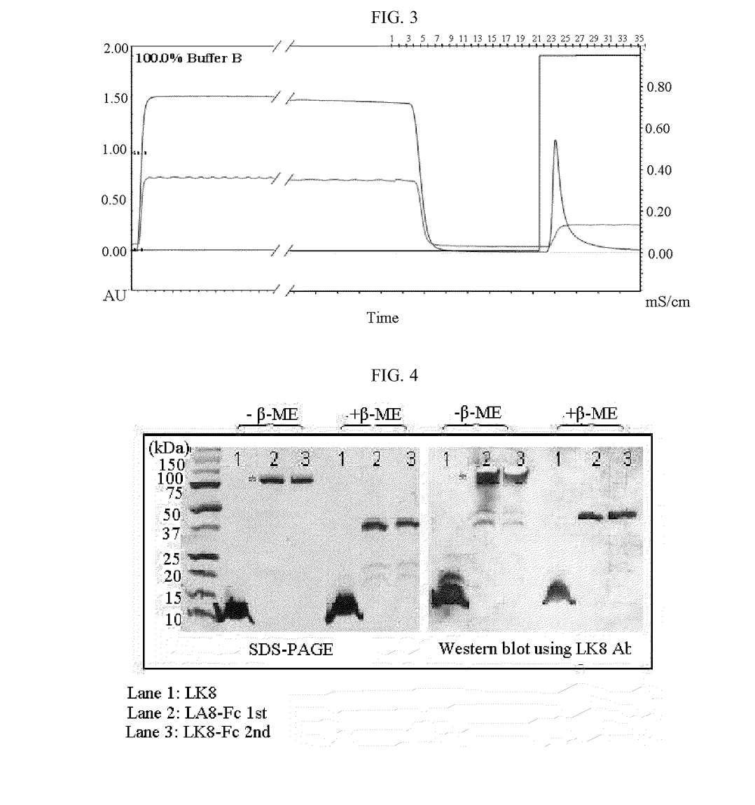

In order to purify the LK8-Fc fusion protein, the CHO/LK8-Fc cell line was spinner cultured in HyQ-SFM-CHO medium in the same manner as in Example 2. As shown in FIG. 2, the cells were cultured while the growth and viability of cells were observed, and on the 6th day of culture, the supernatant was collected through centrifugation. Then, the LK8-Fc fusion protein contained in the supernatant was purified in the following manner. On the basis of the fact that the Fc region of the LK8-Fc fusion protein has affinity for protein G sepharose (Amersham Pharmacia, USA), affinity column chromatography was performed. Specifically, in a binding buffer containing 20-100 mM sodium phosphate (pH 6-8), the LK8-Fc fusion protein contained in the supernatant was bound to the protein G sepharose column, and then it was eluted from the column using a glycine buffer (pH 2-5) (FIG. 3).

The purified LK8-Fc fusion protein was finally dialyzed with PBS, and then the purity thereof was examined by SDS-PAGE using gel with a concentration gradient of 4-20% and Western blotting (FIG. 4). In the electrophoresis results, the molecular weight of the fusion protein was about 37 kDa under a reducing condition and was about 75 kDa under a non-reducing condition. The reason why the molecular weight in the non-reducing condition was about two times higher than that in the reducing condition is because the fusion protein was present as a dimer in the non-reducing condition due to disulfide bonds present in the Fc region of the LK8-Fc fusion protein (FIG. 4).

### Example 4

### Analysis of Ability of LK8-Fc Fusion Protein to Inhibit Endothelial Cell Migration

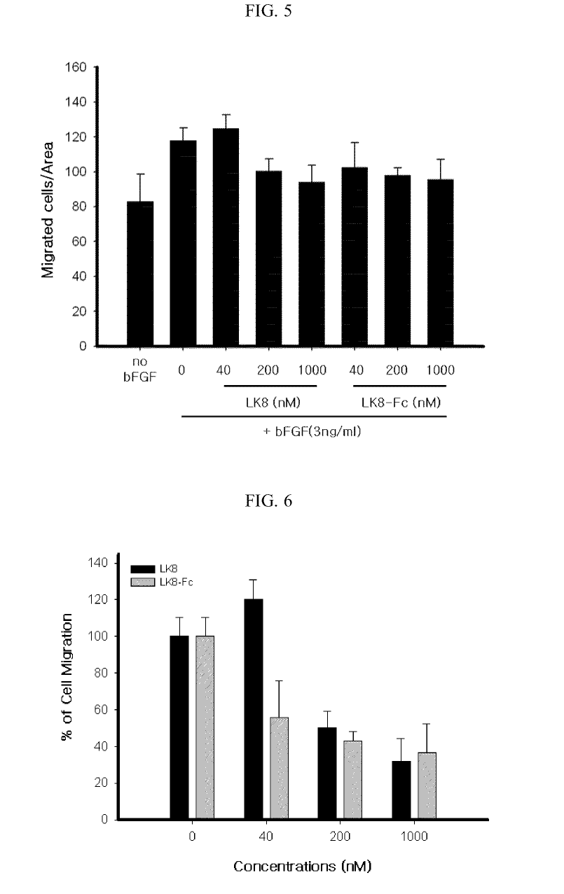

In order to analyze whether the recombinant protein LK8-Fc has angiogenesis inhibitory activity, wounding migration assay was performed in vitro using human umbilical vein endothelial cells (HUVEC; Cambrex, USA) (Kim et al., J. Biol. Chem., 278:29000, 2003). Specifically, HUVEC cells suspended in EGM-2 medium (Cambrex, USA) were placed in each well of a 24-well tissue culture plate coated with 1.5% gelatin and were cultured to a confluency of at least 90%, and then the medium was replaced with 0.1% FBS-containing EBM-2 (Cambrex, USA) medium. After the cells were cultured in the above conditions for about 15 hours, the cells were scratched with a micropipette tip, and the cells detached from the culture plate were removed by washing them twice with PBS. The scratched portion was photographed and marked with a reference line. Scrape-wounded HUVEC monolayers were incubated with bFGF in the presence or absence of LK8-Fc, and the migration of HUVEC into the denuded area was observed over the following 8 h. Then, the inhibition of cell migration was observed by counting the number of cells, which migrated beyond the reference line. The above experiment was repeated three times, and the experimental results are shown in FIGS. 5 and 6

In FIG. 5, the X-axis indicates the kinds and concentrations of treated samples, and the Y-axis indicates the number of cells, which migrated beyond the reference line. FIG. 6 is a graphic diagram showing percentages calculated from the data of FIG. 5. Specifically, FIG. 5 shows the relative inhibition of migration of cells treated with various concentrations of the LK8-Fc fusion protein, in which the relative inhibition was determined by subtracting, from each data, the number of cells migrated in the group treated only with PBS without being treated with the sample, and calculating as percent (100%=the number of migrated cells in the group treated only with bFGF). The LK8 protein was used as a positive control group.

When endothelial cells are treated with bFGF, the migration of the cells is greatly induced. As shown in FIGS. 5 and 6, when the cells were treated with the LK8 protein, the cell migration induced by bFGF was inhibited, and an increase in the concentration of the LK8 protein treated, led to an increase in the inhibitory activity thereof. In the case where the endothelial cells were treated with the LK8-Fc fusion protein at the same molar concentration as that of the LK8 protein, the migration of the HUVEC cells was effectively inhibited to an extent similar to the case of the LK8 protein. Where the cells were treated with each of the LK8 protein and the LK8-Fc fusion protein at a concentration of 1 μM, the LK8 protein-treated group and the LK8-Fc fusion protein-treated group showed endothelial cell migration inhibitory activities of about 68% (p<0.005) and about 64% (p<0.05), respectively, compared to the group treated with bFGF alone.

From the above results, it could be seen that the LK8-Fc fusion protein showed endothelial cell migration inhibitory activity at a level similar to that of the LK8 protein in in vitro conditions.

### Example 5

### Analysis of Ability of LK8-Fc Fusion Protein to Inhibit Angiogenesis In Vivo

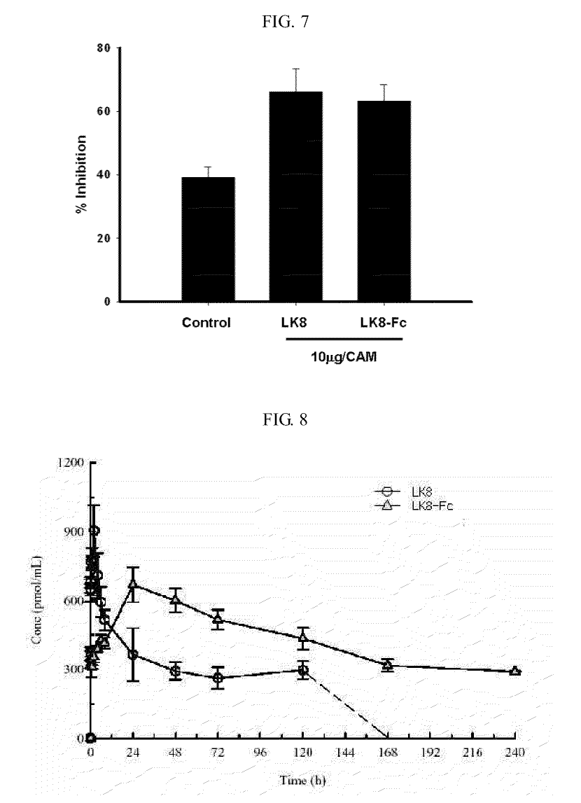

In order to examine whether the LK8-Fc fusion protein inhibits angiogenesis in vivo, the effect of the LK8-Fc fusion protein on angiogenesis in the chorioallantoic membrane (hereinafter, abbreviated as “CAM”) of chick embryos was observed (Kim et al., J. Biol. Chem., 278:29000, 2003). Specifically, the ovalbumin of chick embryos was partially removed, and then a window for protein treatment and observation was made in the chick embryos. Then, the resulting chick embryos were cultured in an incubator at 37° C. for 48 hours. The LK8-Fc fusion protein and the LK8 protein were placed on the Thermanox coverslip (Nunc, USA) and dried, after which each of the proteins was injected into the embryonic CAM, and the embryos were additionally cultured for 48 hours. Then, a fat emulsion was injected into the embryonic chorioallantoic membrane, and angiogenesis around the theramanox was observed. In this example, 60 chick embryos were used per group (FIG. 7).

As a result, in the embryos treated with saline as a negative control group, an angiogenesis inhibition of about 39.2±5.6% was shown. In comparison with this, in the cases treated with 10 μg of each of the LK8 protein and the LK8-Fc fusion protein, respectively, angiogenesis inhibitions of about 66.2% (p<0.05 as compared to the control group) and about 63.2% (p<0.05 as compared to the control group) were observed. That is, it was shown that treatment with each of the samples significantly inhibited angiogenesis, and no difference in effect between the two samples was observed. Therefore, it was confirmed that the LK8-Fc fusion protein showed angiogenesis inhibitory activity not only in in vitro conditions in Example 3, but also in in vivo conditions.

### Example 6

### Pharmacokinetic (PK) Analysis of LK8-Fc Fusion Protein

In order to observe the pharmacokinetics of the LK8-Fc fusion protein, each of the LK8-Fc fusion protein and the LK8 protein was administered to 6-week-old male SD rats (Charles River, Japan) once, and then the concentration of the LK8-Fc fusion protein in blood plasma was measured at various points of time. Specifically, the LK8-Fc fusion protein and the LK8 protein were labeled with FITC (Sigma, USA), and 180 μg of each of the LK8-Fc-FITC and LK8-FITC proteins was injected intramuscularly to SD rats (3 animals per group) once for protein detection. After the administration of the proteins, 200 μA of blood was sampled from the animals through eye bleeding at intervals of 0.017, 0.051, 0.085, 0.17, 0.51, 1, 2, 4, 6, 8, 24, 48, 72, 120 and 168 hr, and then blood plasma was extracted from the blood samples. The concentration of the protein in the blood plasma was determined by measuring absorbance at 490 nm (excitation wavelength of FITC) and 535 nm (emission wavelength of FITC) using a Fluorometer (PerkinElmer, USA) and calculating the protein concentration based on the measured absorbance value using a standard curve (Table 2).

TABLE 2

| | |
|---|---|
| LK8 protein | LK8-Fc fusion protein |
| Conc. (pmol/mL) | Conc. (pmol/mL) |

| Time (h) | mean | SD | mean | SD |
|---|---|---|---|---|
| 0.017 | 643.6 | 11.6 | 366.3 | 28.5 |
| 0.051 | 647.7 | 53.2 | 368.4 | 31.2 |
| 0.085 | 661.0 | 10.9 | 342.1 | 37.4 |
| 0.17 | 649.6 | 27.6 | 347.4 | 46.1 |
| 0.51 | 787.4 | 25.3 | 329.9 | 62.7 |
| 1 | 779.7 | 48.6 | 311.0 | 8.2 |
| 2 | 03.3 | 113.0 | 353.5 | 23.4 |
| 4 | 708.7 | 97.3 | 388.9 | 13.3 |
| 6 | 592.5 | 64.9 | 429.4 | 20.5 |
| 8 | 514.7 | 44.1 | 414.1 | 23.6 |
| 24 | 364.5 | 116.1 | 667.4 | 75.7 |
| 48 | 292.1 | 38.6 | 599.3 | 52.6 |
| 72 | 261.4 | 47.7 | 516.4 | 42.9 |
| 120 | 296.5 | 38.6 | 434.5 | 47.7 |
| 168 | — | — | 317.0 | 27.4 |

The pharmacokinetics of the proteins were analyzed through the data shown in Table 2. As a result, in the group administered with the LK8-Fc fusion protein, the half-life (t_{1/2}) of the LK8-Fc fusion protein was shown to be about 177 hr, and the AUC (0-t) and AUC (inf), indicative of in vivo exposure, were analyzed to be 103,001 h·pmol/mL and 176,759 h·pmol/mL, respectively (Table 3 and FIG. 8). Accordingly, it was confirmed that the half-life of the LK8-Fc fusion protein was increased due to the Fc fusion partner, and thus in vivo bioavailability thereof was significantly increased.

TABLE 3

| Parameter | LK8 protein | LK8-Fc fusion protein |
|---|---|---|
| Cmax (pmol/mL) | 903 | 667 |
| Tmax (h) | 2 | 24 |
| AUC (0-t) (h · pmol/mL) | 40,536 | 103,001 |
| AUC (inf) (h · pmol/mL) | —¹ | 176,759 |
| λz (h⁻¹) | —¹ | 0.00392 |
| t_{1/2} | —¹ | 177 |

¹concentration in serum could not be measured because there was no slope of log line at C120 > C72.

### Example 7

### Inhibition of Solid Tumor Growth by Treatment with LK8-Fc Fusion Protein

A tumor model xenotransplanted with human colon cancer cells was used to observe whether the LK8-Fc fusion protein had an inhibitory effect on the growth of solid cancer. Specifically, about 5×10⁶ LS174T human colon cancer cells (ATCC, USA), cultured in DMEM (GIBCO, USA) supplemented with 10% FBS (GIBCO, USA), were inoculated subcutaneously into the proximal central portion of the back of BALB/c nude mice (Charles River, Japan). At 10th day after the implantation of the colon cancer cells, each of the LK8-Fc fusion protein and the LK8 protein was administered to the mice. The LK8-Fc fusion protein and the LK8 protein were administered at doses of 35 mg/kg/time and 10 mg/kg/time, respectively, such that they were administered at the same molar concentration. In administration schedules, the LK8-Fc fusion protein was administered once at 7-day intervals on the basis of the PK test results obtained in Example 6. For the comparison of efficacy between the LK8-Fc fusion protein and the LK8 protein, animals for administration with the LK8 protein were divided into a group administered with the LK8 protein once at 7-day intervals and a positive control group administered with the protein once a day, in which the administration schedule for the positive control group was confirmed to be effective through the previous experiment. 5 animals were used per group, and the growth of cancer was observed for about one month after the transplantation of the tumor cells. The treatment procedure was continued for 20 days, and the size of tumor was measured once at an interval of 3-4 days. Tumor size was calculated by the formula width²×length×0.52. These experiments were repeated two times with similar results.

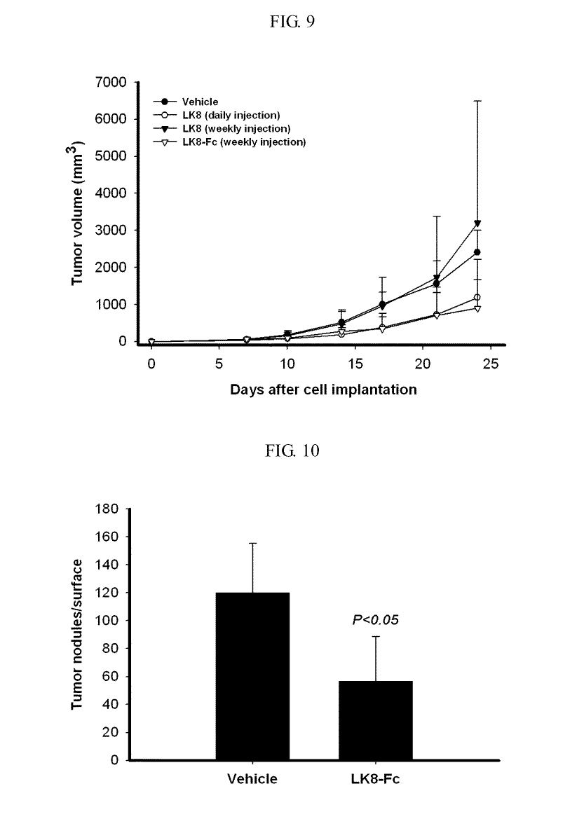

As a result, the growth of tumor was inhibited due to treatment with the LK8 protein and the LK8-Fc fusion protein. Also, in the group administered with the LK8 protein once a day and the group administered with the LK8-Fc fusion protein once at 7-day intervals, a significant tumor growth inhibitory effect compared to that in the control group was observed (FIG. 9). However, in the case where the LK8 protein was administered once at 7-day intervals as in the administration schedule for the LK8-Fc fusion protein, no tumor growth inhibitory effect was observed. Specifically, in the results of observation at 21th day after the implantation of the cell line, the tumor volume of the control group administered with saline was 2409±591 mm³ (±SD) on average, whereas the tumor volume of the group administered with the LK8 protein once a day was 1188±1022 mm³ (±SD), the tumor volume of the group administered with the LK8 protein once at 7-day intervals was 3203±3284 mm³ (±SD), and the tumor volume of the group administered with the LK8-Fc fusion protein once at 7-day intervals was 899±773 mm³ (±SD). That is, in comparison with the mean value of tumor growth rates of the control group administered with saline, the group administered with the LK8 protein once a day showed a tumor growth inhibition of about 50%, and the group administered with the LK8-Fc fusion protein once at 7-day intervals showed a tumor growth inhibition of about 63%. The group administered with the LK8-Fc fusion protein once at 7-day intervals, showed a tumor inhibition similar to that of the group administered with the LK8 protein once a day, and this was attributable to the increased half-life of the LK8-Fc fusion protein.

### Example 8

### Analysis of Metastasis Inhibitory Activity of LK8-Fc Fusion Protein

In order to observe whether the LK8-Fc fusion protein has an inhibitory effect against liver metastasis of a colon cancer cell line, an animal model, obtained by implanting colon cancer cells into the spleen of BALb/c nude mice (Charles River, Japan), was used to observe liver metastasis. Specifically, BALB/c nude mice were anesthetized with ketamine (Sigma, USA), and then, 3×10⁵ LS174T human colon cancer cells were transplanted into the spleen, and after one day, the administration of the LK8-Fc fusion protein was initiated. In the same manner as in the case of the solid cancer model, the protein was administered at a concentration of 35 mg/kg/time once at 7-day intervals on the basis of the PK test results obtained in Example 6. At 14^{th} day after the tumor implantation, the mice were sacrificed, and the liver was taken out. Then, the observation of cancer was performed, and the number of metastasized tumor nodules was counted, thus determining cancer metastasis.

As a result, the number of nodules, produced by metastasis to the liver surface, was 120.3±35.1 (±SD) per unit area in the control group treated with saline, whereas the number was 56.8±31.9 (±SD) in the group treated with the LK8-Fc fusion protein, suggesting that, in the group administered with the LK8-Fc fusion protein, the number of nodules produced by metastasis was significantly reduced compared to that in the control group (FIG. 10).

### INDUSTRIAL APPLICABILITY

As described in detail above, the present invention provides an LK8-Fc fusion protein in which an LK8 protein is fused with the Fc region of human immunoglobulin IgG1. Also, the present invention provides a composition for treating cancer, which contains the LK8-Fc fusion protein. The LK8-Fc fusion protein according to the present invention has not only angiogenesis inhibitory activity leading to anticancer and metastasis inhibitory activities, but also a very long in vivo half-life, and thus can be used as a more efficient and economic cancer therapeutic agent or cancer inhibitor.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. An LK8-Fc fusion protein in which a LK8 protein is fused with the Fc region of human immunoglobulin IgG1.

2. The LK8-Fc fusion protein according to claim 1, which additionally contains an Igκ leader sequence for extracellularly secreting the fusion protein.

3. A gene encoding the LK8-Fc fusion protein of claim 1.

4. A recombinant vector containing the gene of claim 3.

5. Recombinant cells transfected with the recombinant vector of claim 4.

6. The recombinant cells according to claim 5, wherein said cells are animal cells.

7. The recombinant cells according to claim 5, wherein said animal cells are CHO/LK8-Fc cells.

8. A method for preparing LK8-Fc fusion protein, the method comprises: culturing the recombinant cells of claim 5.

9. A composition for treating cancer, which contains the LK8-Fc fusion protein of claim 1.

10. The composition for treating cancer according to claim 9, wherein the cancer is selected from the group consisting of: colorectal cancer, pancreatic cancer, prostate cancer, renal cancer, melanoma, bone metastases of prostate cancer, and ovarian cancer.

11. A composition for inhibiting angiogenesis, which contains the LK8-Fc fusion protein of claim 1.


SEQUENCE LISTING (6 sequences)

SEQ ID NO: 1 - DNA, 258 bp, Homo sapiens
gaacaagactgtatgtttgggaatgggaaaggataccggggcaagaaggcaaccactgttactgggacgccatgccaggaatgggctgcccaggagccccatagacacagcacgttcattccagggacaaataaatgggcaggtctggaaaaaaattactgccgtaaccctgatggtgacatcaatggtccctggtgctacacaatgaatccaagaaaactttttgactactgtgatatccctctctgtgcatcctct

SEQ ID NO: 2 - DNA, 699 bp, Homo sapiens
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatga

SEQ ID NO: 3 - DNA, 34 bp, Artificial Sequence
  Primer
gcggcccagccggccgaacaagactgtatgtttg

SEQ ID NO: 4 - DNA, 32 bp, Artificial Sequence
  Primer
cgggatccagaggatgcacagagagggatatc

SEQ ID NO: 5 - DNA, 26 bp, Artificial Sequence
  Primer
cgggatccgagcccaaatcttgtgac

SEQ ID NO: 6 - DNA, 30 bp, Artificial Sequence
  Primer
tatactcgagtcatttacccggagacaggg